(19) United States
(12) Patent Application Publication
(10) Pub. No.: US 2024/0057868 A1
(43) Pub. Date: Feb. 22, 2024

(54) System for Optically Measuring Vital Parameters

(71) Applicant: Cosinuss GmbH, Munich (DE)

(72) Inventor: Johannes Kreuzer, Munich (DE)

(21) Appl. No.: 18/259,359

(22) PCT Filed: Dec. 23, 2021
(86) PCT No.: PCT/EP2021/087497

§ 371 Date: Jun. 26, 2023

(30) Foreign Application Priority Data
Dec. 24, 2020 (DE) 10 2020 216 571.8

(51) Int. Cl. A61B 5/00

(52) U.S. Cl. CPC A61B 5/0059

(57) ABSTRACT

A system for optically measuring vital parameters, in particular by means of the pulse oximetry method, is disclosed. The system (1) comprises a controller (4) configured to control one or more transmitters of light (2a, 2b, 2c) depending on a control signal from a signal processor (7) in such a way that the signal quality of an incoming signal is maximal, and the controller (4) is furthermore configured to select a wavelength range of the one or more transmitters of light (2a, 2b, 2c) on the basis of context information, the context information being selected from the group comprising movement information, location information, time information, light intensity information.

The present invention relates to a system for the optical measurement of vital parameters according to the subject matter of claim 1.

### STATE OF THE ART

In order to record vital parameters by means of optical sensors, light is radiated into the tissue by means of a light transmitter and the intensity of the light emerging from the tissue is recorded by means of a light receiver. The vital parameters thereby recorded include, for example, the arterial oxygen saturation, which can be determined non-invasively using the pulse oximetry method. Pulse oximetry is based on detecting the reflection of emitted infrared light rays, whereby light shines through the tissue of a body part, a finger, a target, an earlobe or the tissue in the external auditory canal. The light is modulated by the arterial blood so that the oxygen saturation and the pulse rate can be deduced. In addition to oxygenated and deoxygenated hemoglobin, other types of hemoglobin (methaemoglobin, carboxyhemoglobin) or other blood parameters or physiological parameters can be measured, such as blood sugar concentration or body temperature. Pulse oximetry can use either green light (advantageous for detecting brushing frequency during movement) or light in the infrared range (advantageous for detecting arterial blood oxygen saturation). Each light color has different advantages, which differ with regard to various conditions and factors, for example visibility through the human eye, penetration depth of the light into the tissue, susceptibility to interference (motion artifacts), or efficiency.

In the case of a measurement in the external auditory canal, in which the temperature and other parameters are measured simultaneously, the arrangement of the individual sensor elements is not trivial. Light transmitters not only radiate light into the tissue but also generate heat through current flow. A displacement of the sensor elements in the ear canal, for example due to movement, can lead to signal interference, for example because the emitted light signal only partially shines through the tissue. In the case of the conventionally used devices for the optical measurement of vital parameters, which are worn in the external auditory canal, the signal quality can fluctuate considerably depending on environmental conditions (e.g., movement of the wearer of the device, strong scattered light), impairing the detection of the signal and subsequently the significance of the decrease the vital parameters recorded with the signal.

### Presentation of the Invention, Task, Solution, Advantages

Starting from the aforementioned devices of the prior art, the object of the present invention is therefore to provide a holding device which does not have the disadvantages mentioned. There is a need to provide a non-invasive, easy-to-use system for obtaining multiple vital parameter that allows both discrete and continuous acquisition of one or more vital parameters, and which can provide at rest, during physical activity, and under various environmental conditions (e.g., example strong scattered light) reliable measurement data.

In a first aspect, the invention provides a system for the optical measurement of vital parameters. A vital parameter is understood to be a parameter that reflects a basic function of the body of a living being and that either consists of a continuous (e.g., electrocardiogram) or a discrete (e.g., heart rate, blood pressure, body temperature, respiratory rate) measurement result. The system according to the invention comprises one or more transmitters of light with wavelength ranges that differ from one another, a controller for controlling the one or more transmitters of light with wavelength ranges that differ from one another, wherein a driver can be arranged between the controller and the one or more transmitters of light. The system further comprises at least one light receiver for receiving a signal, a signal processor for processing the signal of the one or more transmitters of light detected by the at least one light receiver, wherein a front end can be arranged between the light receiver and the signal processor. The front end can be set up to convert the signal of the one or more transmitters of light detected by the at least one light receiver. The controller according to the invention is set up to control the one or more transmitters of light as a function of a control signal from the signal processor in such a way that the signal quality of an incoming signal is at its maximum, and the controller is also set up to limit a wavelength range of the one or more transmitters of light to be selected based on context information, the context information being selected from the group comprising movement information, location information, time information, light intensity information.

The controller according to the invention is an electronic component and is set up to control the one or more light emitters, the controller according to the invention being set up for controlling and regulating. The term “control” means a process in which an event signal influences a control variable through a control algorithm (control system) implemented in the system. The term “control” means measuring the variable to be influenced (controlled variable) and continuously comparing it with the selected reference variable, with a controller determining a manipulated variable from the control deviation (control difference) and the specified control parameters. The manipulated variable then acts on the controlled variable via the controlled system in such a way that it minimizes the control deviation despite the presence of disturbance variables and the controlled variable assumes a desired time behavior depending on the selected quality criteria. In the further description, the term “regulate” is also subsumed under the term “control” by the controller.

The light receiver according to the invention is a radiation light receiver for measuring electromagnetic radiation, wherein depending on the design of the light receiver radiation of different wavelengths can be detected. For example, the at least one light receiver according to the invention can be designed as a photodiode. The controller is set up to receive a control signal from the signal processor and then to control the one or more transmitters of light in such a way that the signal quality of an incoming signal is maximum. The signal quality describes the quality of the signal received by the light receiver, for example the signal-to-noise ratio. The signal-to-noise ratio is a measure of the technical quality of a useful signal that is superimposed by a noise signal and is defined as the ratio of the mean power of the useful signal to the mean noise power of the interference signal. The controller controls the one or more transmitters of light, for example by changing their intensity or the spectrum of the wavelength range. In addition, the controller is set up to use context information to select a wavelength range of the one or more transmitters of light. Such context information includes movement information, which can be conveyed to the controller, for example, via signals from inertial sensors (acceleration and rotation sensors), or location information, which can be conveyed, for example, via signals from a GPS receiver. Time information can be conveyed to the controller via signals from a corresponding hardware or software clock, while the light intensity information is conveyed to the controller via signals generated by the light receiver.

The system for measuring vital parameters according to the invention can advantageously allow optimal measurements under a wide variety of environmental conditions by adapting the one or more transmitters of light with regard to their wavelength range in order to ensure optimal signal quality of the incoming signal. For example, in the event of strong movement of the system arranged on the body of a living being, the controller can cause the light emitter(s) to emit light with a shorter wavelength, which penetrates less deeply into the tissue, making it less susceptible to interference signals in order to improve the signal quality of the incoming signal to the signal processor improve signal. When determining the vital parameter “heart rate”, the light emitter with the appropriate wavelength can be selected depending on the context information resting state/movement, namely red light in the resting state and green light with movement. For example, the wavelength range can be adapted to the reduced stray light conditions in the evening or at night (for example reduced light intensity, measurement in the red/infrared range).

In a further embodiment, the system for the optical measurement of vital parameters can be arranged in the external auditory canal. The system designed in this way advantageously enables both continuous and discontinuous detection of a large number of vital parameters. In particular, continuous detection under everyday conditions is advantageously possible, since the sensors of the system located in the external auditory canal allow comprehensive measurements without restricting the use of, for example, the arms and legs of a wearer of the system, or requiring certain environmental conditions (absence of stray light, shielded Measurement).

In a preferred development of the system according to the invention, the controller can be set up to control a multi-wavelength light transmitter. A multi-wavelength light emitter can be designed, for example, in the form of a light emitting diode (LED) which emits white light; in this case, wavelength ranges in the red or green range can be defined by appropriate filtering. The multi-wavelength light transmitter can preferably be in the form of a double or triple wavelength light transmitter. In particular, it can be expedient to provide several transmitters of light combined in a multi-wavelength light transmitter with wavelength ranges that differ from one another in an optical unit. In this case, the controller can be set up to simultaneously control a plurality of transmitters of light with wavelength ranges that differ from one another, in order, for example, to emit light in the red/infrared wavelength range at the same time. In this way, different vital parameters can advantageously be measured and determined simultaneously. In a preferred implementation of the system according to the invention, the controller can be set up to control a number of transmitters of light with wavelength ranges that differ from one another in a chronological sequence. In particular, different parameters can be measured almost simultaneously and continuously due to the close time sequence of optical signals from the various transmitters of light. In a particularly advantageous manner, the wavelength ranges of the plurality of transmitters of light with wavelength ranges that differ from one another cannot overlap. In particular, conventional LEDs meet the basic requirements by emitting narrow-band, almost monochromatic light, so that costly optical filters to limit the wavelength of the light can be dispensed with.

In a further embodiment, the system according to the invention can comprise at least two light receivers for detecting light emissions with non-overlapping wavelength ranges. The light receiver can preferably be a photodiode, in which case the photodiode can preferably be a silicon photodiode and in particular a silicon PIN photodiode. A transimpedance amplifier or a transimpedance converter can preferably be connected downstream of the photodiode, which converts the photocurrent generated by the photodiode into a measurement voltage proportional thereto. The photocurrent is in turn proportional to the luminous flux received. The photodiode can advantageously be characterized by the lowest possible noise, high sensitivity at the corresponding wavelength, low sensitivity to temperature changes, humidity and movement, and a low operating voltage.

In a further implementation of the system according to the invention, the vital parameters can be selected from the group comprising oxygen saturation, pulse rate, pulse rate variability, cardiovascular parameters, respiratory rate, body temperature. The oxygen saturation (sO2) indicates what percentage of the total hemoglobin in the blood is loaded with oxygen. The pulse rate and the pulse frequency variability are variables that describe the pulse of a living being, i.e., the recurring pressure and volume fluctuations that are generated in the vascular system by the systolic blood output of the heart. From a continuously measured pulse curve, other cardiovascular parameters such as cardiac output (stroke volume×heart rate), cardiac index (cardiac output related to body surface area) and respiratory rate (from pulse amplitude variability) can be estimated. Depending on the presence of additional sensors, for example a temperature sensor, the body temperature can be recorded. A large number of vital parameters can be measured continuously or almost continuously via the system according to the invention without restricting the mobility of the wearer of the system.

In a further embodiment of the system according to the invention, the controller can be set up to control the one or more transmitters of light with different wavelength ranges to emit light in the wavelength range from 600 nm and 750 nm and/or from 750 nm to 1000 nm and/or from 450 nm and 590 nm, preferably for the emission of light in the wavelength range from 640 nm to 700 nm and/or from 880 nm to 960 nm and/or from 530 nm to 570 nm. In particular, the system according to the invention is designed to use light in the red and infrared wavelength ranges for pulse oximetry measurement of oxygen saturation and in the green wavelength range for detecting the pulse rate/pulse curve.

In a preferred development, the signal processor can be set up to select a signal from the incoming signals depending on a selection criterion, the selection criterion being selected from the group consisting of pulse shape, signal-to-noise ratio, AC/DC ratio, artifact superimposition. The quality of the signal can be determined from the shape of the pulse curve (rate of pressure rise and fall, position, and extent of the incision) as well as from the signal-to-noise ratio (signal frequency/interference frequency). In the case of pulse oxymetric measurements, the ratio of the alternating component to the direct component is particularly important Significance: to determine the oxygen saturation, the alternating component (pulsation), which is mainly caused by the arterial blood, is converted into the direct component from the absorption of e.g., tissue and venous blood related. The actual recorded signals can be amplified by signal amplification and the use of band-pass filters (low-pass filters). A further separation of signal and noise signal/artefact and thus an exact determination of the actual signal can advantageously be achieved mathematically using interference reference signals, with the interference reference signals regularly generated for saturation values of 0 to 100% being subtracted from the signal in pulse oximetry (internal reference signal). Signals can also be evaluated mathematically with regard to their synchronicity and weighted accordingly; Another method for selecting an optimal signal uses a frequency criterion (comparison of the measured pulse frequency with the alternating component detected during pulse oximetry). Using the selection from various selection criteria, the signal processor can optimize the signal and, in a particularly preferred implementation, adapt the control signal to the selection criterion, i.e., send a corresponding control signal to the controller.

In a further aspect, the invention comprises a method for the optical measurement of vital parameters, comprising the steps of emitting at least one light signal with a defined wavelength, the corresponding light signal being emitted with a defined period of time, or the emitting of a plurality of light signals with defined, different wavelengths takes place in a defined time period and sequence. The step of emitting the at least one light signal is followed by receiving the light signal after the tissue has passed through the at least one light receiver. The light signal received is first processed by the signal processor in the following steps and evaluated with regard to the signal quality of the incoming signal. control step, see above for definition of term), a control signal is sent to the controller to maximize the signal quality. in a dem After the selection step preceding the first step of the transmission, the controller can select the corresponding transmitter of light on the basis of context information, for example the time of day.

### BRIEF DESCRIPTION OF THE FIGURES

In the following, the system for the optical measurement of vital parameters is described by way of example and not conclusively in a particular embodiment with reference to the attached FIGURE.

The particular embodiment only serves to explain the general inventive idea, but it does not limit the invention.

FIG. 1 shows a block diagram of the system according to the invention for the optical measurement of vital parameters (1).

### PREFERRED EMBODIMENT OF THE INVENTION

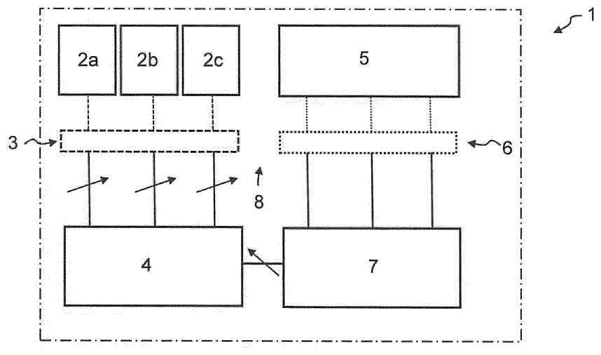

FIG. 1 shows the system for the optical measurement of vital parameters (1) as a block diagram. In the illustrated embodiment, the system (1) comprises three transmitters of light (2a, 2b, 2c), which, for example, emit light with the respective wavelength range infrared (750 nm to 1000 nm), red (600 nm to 750 nm) and green (450 nm to 590 nm). The system also includes a controller (4) which can control or regulate the transmitters of light (2a, 2b, 2c). As explained in the general description, the term “regulate” is also subsumed under the term “control” to describe the present invention. In the present case, a driver (3) is arranged between the controller (4) and the three transmitters of light (2a, 2b, 2c).

A light receiver (5) is arranged on the receiving side of the system (1) according to the invention, which is connected to a signal processor (7) via a so-called front end (6) for signal conversion. The controller (4) is configured to at least one of the three transmitters of light (2a, 2b, 2c) in Depending on the control signal (8), which is sent from the signal processor (7) to control so that the signal quality of the incoming signal is maximum. The controller (4) is also configured so that it can select a wavelength range of at least one of the three transmitters of light (2a, 2b, 2c) based on context information, the context information being, for example, movement information of the carrier of the system (1) according to the invention. For example, a system (1) inserted in the external auditory canal of a wearer can detect the presence of physical activity, for example via acceleration sensors additionally present in the system, so that the controller (4) selects the one with the shorter wavelength range from the existing transmitters of light, in this case the transmitter of light within the green wavelength range. When determining the vital parameter “heart rate”, the light emitter with the appropriate wavelength can be selected depending on the context information resting state/movement, namely red light in the resting state and green light with movement.

### LIST OF REFERENCE SYMBOLS

1 System for the optical measurement of vital parameters
2 Transmitter of light with different wavelength ranges (2a, 2b, 2c)
3 Driver
4 Controller
5 Light receiver
6 Front-end
7 Signal processor
8 Control signal emanating from the signal processor

1. A system for the optical measurement of vital parameters, comprising
one or more transmitters of light with different wavelength ranges,
a controller for controlling the one or more transmitters of light with different wavelength ranges, wherein a driver may be arranged between the controller and the one or more transmitters of light,
at least one light receiver for receiving a signal,
a signal processor for processing the signal of the one or more transmitters of light detected by the at least one light receiver, wherein a front end can be arranged between the light receiver and the signal processor, the controller being set up to detect the one or more transmitters of light depending on a control signal from the signal processor so that the signal quality of an incoming signal is at its maximum, the controller being set up to select a wavelength range of the one or more transmitters of light based on context information, the context information being selected from the group comprising movement information, location information, time information, light intensity information.

2. The system according to claim 1 for the optical measurement of vital parameters in the external auditory canal of a living being.

3. The system according to any one of claim 1 or 2, in which the controller is set up to control a multi-wavelength light transmitter, the multi-wavelength light transmitter preferably being in the form of a double or triple wavelength light transmitter.

4. The system according to any one of claim 1 or 2, in which the controller is set up to control a plurality of transmitters of light with wavelength ranges which differ from one another in a time sequence.

5. The system according to any one of claim 3 or 4, wherein the wavelength ranges of the plurality of light emitters having different wavelength ranges from each other do not overlap.

6. The system according to claim 5, comprising at least two light receivers for detecting light emissions with non-overlapping wavelength ranges.

7. The system according to any one of the preceding claims, wherein the vital parameters are selected from the group comprising oxygen saturation, pulse rate, pulse rate variability, cardiovascular parameters, respiratory rate, body temperature.

8. The system according to any one of the preceding claims, wherein the controller is set up to control the one or more transmitters of light with different wavelength ranges to emit light in the wavelength range from 600 nm and 750 nm and/or from 750 nm to 1000 nm and/or from 450 nm and 590 nm, preferably for the emission of light in the wavelength range from 640 nm to 700 nm and/or from 880 nm to 960 nm and/or from 530 nm to 570 nm.

9. The system according to any one of the preceding claims, wherein the signal processor is set up to select a signal from the incoming signals as a function of a selection criterion, the selection criterion being selected from the group comprising pulse shape, signal-to-noise ratio, AC component-DC component ratio, and artifact-overlay.

10. The system according to claim 9, wherein the control signal is adjusted according to the selection criterion.
